(19) United States
(12) Patent Application Publication
(10) Pub. No.: US 2023/0309826 A1
(43) Pub. Date: Oct. 5, 2023

(54) Ophthalmic Imaging Systems, Methods, and Computer-Readable Media

(71) Applicant: OPTOS PLC, Dunfermline (GB)

(72) Inventor: Praveen Ashok, Dunfermline (GB)

(73) Assignee: OPTOS PLC, Dunfermline (GB)

(21) Appl. No.: 17/712,477

(22) Filed: Apr. 4, 2022

(51) Int. Cl. A61B 3/12; A61B 3/10; A61B 3/14

(52) U.S. Cl. CPC A61B 3/1225; A61B 3/102; A61B 3/14

(57) ABSTRACT

Aspects of the present invention relate to an ophthalmic imaging system for imaging a target on a posterior segment of a patient's eye, the imaging system comprising: a light source configured to emit a light beam for imaging the target; an imaging device for tracking a position of an anterior segment of the patient's eye; and a controller configured to determine a movement of the target in dependence on the tracked position of the anterior segment of the patient's eye; wherein the controller is further configured to adjust an angle of incidence of the light beam that contacts the posterior segment in dependence on the determined movement of the target.

### FIELD

Example aspects herein generally relate to ophthalmic imaging, and, more particularly, to imaging a location on a posterior segment of a patient's eye and/or a location on a retina, and compensating for movements of an eye during retinal imaging or posterior segment imaging.

### BACKGROUND

Optical coherence tomography (OCT) is a non-invasive imaging method used to generate cross-sectional images of tissue. OCT imaging is commonly used in ophthalmology to generate cross-sectional images of a patient's retina. This can be useful as the cross-sectional images of the retina allow a clinician to gain an understanding of the retinal tissue of a patient's eye such that pathogens can be identified in the retina.

Typically, OCT imaging is performed over a period of around one to five seconds depending on the imaging protocol. However, the time of capture may increase depending on the imaging modality. For example, during optical coherence tomography angiography (OCT-A) the imaging time may increase significantly as multiple b-scans of a target are required to be taken.

During image acquisition in an OCT scan the patient's eye is constantly moving which can lead to inaccuracies, noise and imaging artefacts in the resultant image data. Movement of the patient's eye may be a result of involuntary movements of the eye. This problem is exacerbated in OCT scans where the imaging protocol takes longer to complete. For example, image acquisition of a target in an OCT-A scan may last five seconds or longer over which time a patient's eye may be constantly moving thereby causing inaccuracies and artefacts in the resultant OCT image.

Over time techniques have been developed to account for involuntary eye movements of a patient's eye during an OCT scan. For example, post processing algorithms may be used to process the image data acquired during an OCT scan to remove artefacts from the image data caused by involuntary movements of the patient's eye.

Closed loop retinal eye tracking may also be used to compensate for involuntary eye movement during an OCT scan in real time. For example, Scanning Laser Ophthalmoscope (SLO) imaging may be used to capture en-face images of the retina such that the movement of the patient's retina may be tracked. This approach typically employs a separate SLO imaging system, in addition to an OCT imaging system, to generate SLO images of the retina of the patient at the same time as performing OCT scans of a target in the eye. In this system the SLO imaging system may be used to monitor movements of the patient's retina such that the involuntary movements of the patient's eye can be monitored and compensated for during an OCT scan.

### SUMMARY

According to an example aspect herein, an ophthalmic imaging system is provided for imaging a target location on a retina of a patient's eye, the imaging system comprising: a light source configured to emit a light beam towards the target location on the retina; an imaging device configured to capture images of an anterior segment of the patient's eye; and a controller configured to determine a movement of the target location in dependence on the captured images of the anterior segment of the patient's eye; wherein the controller is further configured to adjust an angle of incidence of the light beam that contacts the retina based on the determined movement of the anterior segment such that the light beam tracks the movement of the target location on the retina.

According to an example embodiment herein, the ophthalmic imaging system acquires high resolution images of a target location on the posterior segment or retina of a patient's eye by adjusting the imaging light beam to track the imaging target such that the imaging beam follows the imaging target. Adjusting the light beam to follow the target compensates for movements of the patient's eye during acquisition of images of the target. This in turn improves the signal to noise ratio in the acquired images and furthermore substantially removes artefacts in the resultant images of the target caused by eye movements during image acquisition. The controller may be configured to track the position and/or movements of the anterior segment of the patient's eye in dependence on the captured images of the anterior segment of the eye.

The target, target location or location on the posterior segment or retina may be one or more of a point, an area, a region or a line to be imaged by the imaging system. Furthermore, the imaging target on the retina may be a single point, area or region or the imaging target may be a plurality of points, areas or regions. For example, the position of the target location may be updated once images of a given area have been captures. For example, the target location may be multiple points and A-scans or B-scans may be acquired at each point such that images of a region of the retina are acquired.

In an embodiment the ophthalmic imaging system may further comprise an optical coherence tomography, OCT, imaging module, wherein the OCT imaging module comprises: an OCT interferometer configured to generate an interference signal by combining: a reflected light beam received from the location on the retina via a sample arm of the OCT imaging module with a light beam received from a reference arm of the OCT imaging module; and an OCT detector for generating OCT image data from the generated interference signal indicative of the target.

The OCT imaging module of the ophthalmic imaging system may comprise a scanning laser ophthalmoscopy, SLO, detector configured to receive a portion of the reflected light beam from the sample arm to generate SLO image data of the retina. A beam splitter may be located in the sample arm to split or tap the portion of the reflected light beam from the main reflected light beam in the sample arm prior to the main reflected light beam entering the interferometer. Such splitting/tapping off of a portion of the reflected light beam prior to the reflected light beam entering the interferometer allows the SLO detector to acquire SLO images of the posterior segment and/or of the patient's eye. The SLO images may be images of the retina such as ultra-widefield images of the retina. The SLO images may be used to calibrate the ophthalmic imaging system to determine a relationship between the tracked position of the patient's pupil and the resultant movement of the target on the retina.

Furthermore, the SLO image may be used to set a scan location prior to performing an OCT scan of the imaging target. The controller may be configured to set a scan location on the retina for the light beam to image the target or location on the retina in dependence on the generated SLO image data.

The controller may be configured to acquire a first scanning laser ophthalmoscopy, SLO, image of the posterior segment of the eye or of the retina in a first position and a second scanning laser ophthalmoscopy, SLO, image of the posterior segment of the eye or of the retina in a second position. The controller may be further configured to track a position of the anterior segment of the eye as the eye moves from the first position to the second position. The controller may be further configured to determine a relationship between the tracked position of the anterior segment of the eye and the position of the posterior segment of the eye or retina by comparing the first and second SLO images with the tracked position of the anterior segment of the eye. Tracking the anterior segment of the eye may comprise acquiring a first camera image of the anterior segment in a first position and a second camera image of the anterior segment in a second position. Beneficially, the first and second SLO images of the retina may be used to calibrate the ophthalmic imaging system such that the relationship between the position of the patient's pupil and the resultant position of the imaging target may be determined prior to commencing imaging of the target.

In one embodiment the controller may be configured to compare a movement of an anatomical feature on the posterior segment or retina from a first position in the first SLO image and to a second position in the second SLO image with the tracked position of the anterior segment of the eye to determine the relationship between the position of the anterior segment of the eye and the retina or target location located on the retina. The anatomical feature may be used as a landmark on the SLO images to determine a movement of the retina for a measured pupil displacement. The anatomical feature may be any one of: a pathogen, a blood vessel, an optic nerve, a discoloured area, the optic nerve or any other feature located on the patient's retina.

In an embodiment the ophthalmic imaging system may be configured to measure an axial length of the patient's eye. The axial length may be, for example, a distance from the anterior segment to the posterior segment or from the anterior segment to the retina. The controller may be configured to determine a relationship between a tracked movement of the anterior segment of the patient's eye and a resultant movement of the retina based on the axial length of the patient's eye.

In another embodiment the imaging device may be a camera. The camera may be configured to capture images of the interior segment and/or to generate anterior segment image data of the anterior segment of the patient's eye. The controller may be configured to track movements of the anterior segment of the patient's eye in dependence on the captured images and/or based on the generated anterior segment image data.

The anterior segment of the patient's eye is or includes, on one example herein the patient's pupil, iris, both, and/or another portion of an eye's anterior segment. The posterior segment of the patient's eye may be the entirety or at least a portion of the patient's retina. The target may be a target location on the posterior segment of the patient's eye. The target may be a target location located on the patient's retina.

In accordance with a further example herein there is provided an ophthalmic imaging system for imaging a target or a location on a posterior segment of a patient's eye or retina, the ophthalmic imaging system comprising: an ophthalmic coherence tomography, OCT, imaging module configured to acquire OCT images of the target by emitting a beam of light that is incident on the target; a pupil imaging module for tracking a position of the patient's pupil or for capturing images of the patient's pupil while the OCT imaging module acquires the OCT images; a scanning laser ophthalmoscope, SLO, imaging detector for generating a SLO reference image; and a controller configured to set a scan location of the beam of light in dependence on the generated SLO reference image; wherein the controller is further configured to determine a relative movement of the target in dependence on the tracked position of the patient's pupil and to adjust an angle of incidence of the beam of light that contacts the target in dependence on the determined relative movement of the target to compensate for movements of the patient's eye during OCT image acquisition.

According to a further example aspect herein there is provided a method of compensating for movements of a patient's eye during imaging of a location on a posterior segment of the patient's eye or retina, the method comprising: capturing images of an anterior segment of the patient's eye; determining a movement of the location in dependence on the captured images of the anterior segment; and adjusting an angle of incidence of a light beam directed to the retina for imaging the location on the retina in dependence on the determined movement of the location to compensate for movements of the patient's eye during imaging.

The method may comprise tracking the position of the anterior segment whilst simultaneously determining a movement of the target, although this example is not limiting. The method may comprise adjusting the angle of incidence of the light beam in real-time such that the light beam tracks or follows the imaging target on the patient's retina.

The method may comprise acquiring a first scanning laser ophthalmoscopy, SLO, image of the retina in a first position and acquiring a second SLO image of the retina in a second position. The method may further comprise tracking a position of the anterior segment of the eye and determining a relationship between the tracked position of the anterior segment of the eye and a position of the retina by comparing the first and second SLO images with the tracked position of the anterior segment of the eye.

The method may comprise acquiring a first SLO image of the posterior segment or retina in a first position and a first camera or pupil image of the anterior segment in the first position; acquiring a second SLO image of the posterior segment or retina in a second position and a second camera or pupil image of the anterior segment in the second position; and comparing a movement of the retina or posterior segment in the first and second SLO images with the tracked movement of the anterior segment in the first and second camera images to determine a relationship between movement of the anterior segment of the eye and a resultant movement of the target and/or retina.

In an embodiment comparing a movement of the retina or the first and second SLO images with the tracked movement or position of the anterior segment may comprise comparing a movement of an anatomical feature on the retina from a first position in the first SLO image to a second position in the second SLO image with the tracked position of the anterior segment of the eye in the first and second camera images to determine the relationship between the tracked position of the anterior segment of the eye and the retina.

The method may further comprise moving a fixation target from a first fixation position to a second fixation position to steer the patient's eye to move the retina from the first position to the second position. Moving the fixation target from the first fixation position to the second fixation position may be performed during a calibration procedure prior to imaging the target.

In an embodiment the method may comprise acquiring a reference SLO image of the posterior segment or retina of the patient's eye and selecting a scan position for imaging the location or target on the retina in dependence on the acquired reference SLO image. The SLO image may be an image of the patient's retina. The scan location may be an anatomical feature or imaging target on the retina.

Acquiring the reference SLO image may comprise splitting the reflected light beam. Splitting the reflected light beam may comprise splitting the reflected light beam in a sample arm of an optical coherence topography imaging module. Splitting the reflected light beam may comprise splitting the reflected light beam prior to combining the reflected light beam with a reference arm light beam. Splitting the reflected light beam may comprise splitting the reflected light beam using a beam splitter located between the patient's eye and an interferometer. The beam splitter may be located in the sample arm. Splitting the reflected light beam may comprise splitting a reflected portion of the light beam wherein the light beam is reflected by the imaging target on the patient's retina.

Tracking a position of the anterior segment may comprise detecting a reference position of the patient's eye and detecting a movement of the anterior segment from the reference position. Capturing images of the anterior segment may comprise detecting a reference position of the patient's eye and detecting a movement of the anterior segment from the reference position. The reference position may be a position in which the patient is gazing directly at a fixation target.

In an embodiment determining a movement of the location may comprise tracking the position of the anterior segment and comparing the tracked position of the anterior segment with a look-up table to determine the movement of the location. The look-up table may be a table of anterior segment or pupil displacements with an expected posterior segment, retinal or target displacement for an average dimensioned eye.

The method may further comprise measuring an axial length of the patient's eye between the anterior segment and the posterior segment or retina and determining a relationship between the captured images or tracked movement of the patient's eye and the movement of the location in dependence on the measured axial length. The method may comprise determining a displacement of the target in dependence on the axial length of the patient's eye and the displacement of the patient's pupil.

In one embodiment the method may comprise performing closed-loop feedback to adjust the angle of incidence of the light beam in dependence on the determined movement of the location or on the tracked position of the anterior segment.

Tracking the position of the anterior segment may be performed in real-time. Furthermore, tracking the position of the anterior segment may comprise tracking the position of the patient's pupil. The target may be an imaging target or target location located on the patient's retina. Tracking the position of the anterior segment may be performed simultaneously with imaging the target on the posterior segment or retina.

In one example embodiment determining the movement of the location may comprise processing captured images of the anterior segment of the eye to track a position of the patient's pupil. Processing the captured images may comprise image processing techniques to identify and track the location of the patient's pupil from the captured images.

According to a further example embodiment there is provided a method of compensating for movements of a patient's eye during imaging of a target on a posterior segment of the patient's eye, the method comprising: tracking a position of an anterior segment of the patient's eye; determining a movement of the target in dependence on the tracked position of the anterior segment; and adjusting an angle of incidence of a light beam that contacts the posterior segment in dependence on the determined movement of the target such that the light beam tracks the determined movement of the target.

According to a still further example embodiment herein, there is provided a method of compensating for movements of a patient's eye during retinal imaging of a target within the patient's eye or on the patient's retina, the method comprising: tracking movement of the patient's pupil or an anterior segment of the patient's eye; determining a movement of the patient's retina or a target on the patient's retina in dependence on the tracked movement of the patient's pupil or anterior segment; and compensating for the determined movement of the target by controlling an angle of an imaging light beam such that the imaging light beam tracks the determined movement of the target.

The present inventor has also devised, in accordance with a still further example aspect herein, a computer program comprising computer-readable instructions which, when executed by a computer, cause the computer to perform a method according to at least one of the aforementioned methods, aspects and/or embodiments. The computer program may be stored on one or more non-transitory computer-readable storage media (e.g. a CD, a hard drive or a memory stick) or carried by a signal (e.g. an Internet download).

Within the scope of this application, it is intended that the various aspects, embodiments, examples and alternatives set out in the preceding paragraphs, in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

Example embodiments will now be explained in detail, by way of non-limiting example only, with reference to the accompanying figures described below. Like reference numerals appearing in different ones of the figures can denote identical or functionally similar elements, unless indicated otherwise.

FIG. 1 is a schematic diagram of an ophthalmic imaging system according to an example embodiment herein;

FIG. 2 is a flow diagram outlining a method of compensating for movement of a patient's eye during image acquisition using the ophthalmic imaging system of FIG. 1, according to an example embodiment herein;

FIGS. 3a and 3b are first and second images of an anterior portion of a patient's eye in a first and second position, respectively;

FIG. 4a is a schematic sectional view of the patient's eye in the first position of FIG. 3a with a light beam imaging a target on the posterior portion of the eye;

FIG. 4b is a schematic sectional view of the patient's eye in the second position of FIG. 3b with a light beam missing the target location;

FIG. 4c is a schematic sectional view of the patient's eye in the second position of FIG. 3b with a light beam adjusted to follow the target on the posterior portion of the eye;

FIG. 5 is a schematic diagram of an ophthalmic imaging system comprising an OCT imaging module, according to another example embodiment herein;

FIG. 6 is a flow diagram outlining a method of acquiring OCT images of a patient's eye using the ophthalmic imaging system of FIG. 5, according to an example embodiment herein;

FIG. 7 is a flow diagram outlining a method of determining a relationship between a position of a patient's pupil and a resultant position of the patient's retina, according to an example embodiment herein; and

FIG. 8 is an example hardware implementation of an apparatus that can operate as an ophthalmic imaging system according to an example embodiment herein.

### DETAILED DESCRIPTION

In general terms example embodiments herein relate to an ophthalmic imaging system for imaging a target on a posterior segment of a patient's eye such as a target on a patient's retina, and to methods and computer-readable media that operate in accordance therewith. According to one example embodiment herein, the imaging system comprises a pupil tracker or imaging device arranged to capture images of the anterior segment of the eye such that the movement of an anterior segment of the patient's eye, such as the patient's pupil, during image acquisition of the target. The imaging system further comprises a light source configured to emit an imaging beam for imaging the target on the patient's retina. The ophthalmic imaging system further comprises a controller configured to receive movement data indicative of a tracked position of the patient's pupil from the imaging device. The controller determines a resultant movement of the target or movement of the patient's retina in dependence on the tracked movement of the patient's pupil. Furthermore, the controller adjusts an angle of the imaging beam to track the target on the retina as the patient's eye moves to compensate for involuntary movements of the patient's eye during image acquisition.

Tracking movements of the patient's pupil using an imaging device, such as a camera, enables the ophthalmic imaging system to determine the movement of a target on the patient's retina based on movements of the patient's pupil. In one example embodiment herein, Tracking the position and movement of a patient's pupil may be performed using a camera or the like. The imaging beam may be controlled or adjusted by the controller to track a target on the patient's retina based on the determined movements from the position of the pupil.

This can be useful in optical coherence tomography angiography to address situations where multiple images or B-scans of a target are used to complete the imaging process and wherein movements of the patient's eye can create artefacts and noise in the resultant images. Furthermore, in an example embodiment herein, the imaging device may be a camera used to capture images of a patient's pupil, thereby obviating a need, in at least some embodiments, for a separate SLO imaging system or the like to image a patient's retina during acquisition of OCT images, although a SLO imaging system can be employed in other embodiments.

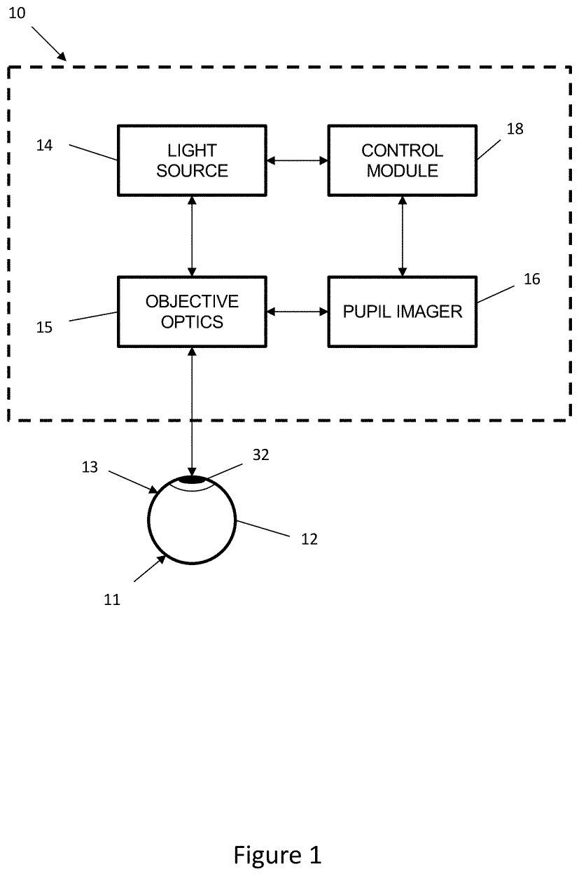

FIG. 1 shows a schematic diagram of an ophthalmic imaging system 10 for imaging a target within a patient's eye 12, according to an illustrative example embodiment herein. The imaging system 10, according to one example embodiment herein, may be an optical coherence tomography, OCT, imaging system for acquiring optical coherence tomography, OCT, images of a location or target (not shown in FIG. 1) located on a posterior segment or retina 11 of the patient's eye 12. The ophthalmic imaging system comprises a light source 14 configured to emit a light beam along an optical path for imaging the target on the posterior segment or retina 11 of the eye 12. The light beam emitted by the light source 14 passes through the objective optics 15 prior to entering the patient's eye 12 via the patient's pupil 32. The objective optics 15 can control the optical path of the light beam such that the light beam can scan the patient's retina and/or image the target on the posterior segment of the patient's eye 12, such as a target on the patient's retina 11. The light source 14 may be used as part of an OCT and/or a scanning laser ophthalmoscope, SLO, imaging system for imaging the target.

The ophthalmic imaging system 10 further comprises a pupil imager 16, or imaging device such as a camera, for imaging an anterior segment 13 of the patient's eye 12. The pupil imager 16 may be a camera used to align a patient's eye 12 within the ophthalmic imaging system 10. The pupil imager 16 is configured to capture images of the anterior segment 13 of the patient's eye 12 so movements of the anterior segment 13 of the eye 12, such as movements of the pupil 32, can be tracked by the control module 18. For example, in one embodiment herein the pupil imager 16 can capture images of the patient's pupil 32 or the patient's iris in real-time such that real-time movements of the anterior segment 13 of the patient's eye 12 can be tracked during image acquisition of the imaging target.

The pupil imager 16, in one example embodiment herein, is optically coupled to the objective optics 15 such that reflected light can be conveyed from the patient's eye 12 to the pupil imager 16 via the objective optics 15 as represented in FIG. 1.

Alternatively, the pupil imager 16 can be optically aligned with the patient's eye 12 such that reflected light travels directly from the patient's eye 12 to the pupil imager 16 such that the pupil imager 16 can generate pupil image data of the anterior segment 13.

Pupil image data, for example the captured images of the anterior segment 13 of the patient's eye 12, captured by the pupil imager 16 is provided to a control module 18. The control module 18 or controller is a component of the ophthalmic imaging system 10 and is coupled to the pupil imager 16 and the light source 14, for controlling those components and for exchanging data and information therebetween. The control module 18 is configured to receive images and/or pupil image data of the anterior segment 13 of the patient's eye 12 from the pupil imager 16. The control module 18 can track movements of the anterior segment 13 of the patient's eye 12 and, in particular, track movements of the patient's pupil in real-time in dependence on (i.e., based on) the received pupil image data and/or the captured images.

The control module 18 is configured to determine, in dependence on the captured images of the anterior segment 13 of the eye 12, a resultant movement of the retina 11. For example, the control module 18 can determine a movement of an imaging target on the patient's retina 11 in dependence on a movement of the pupil 32. As the patient moves their eye 12 the anterior segment 13 of the eye 12 also moves. This in turn causes a movement of the patient's retina 11 due to the patient's eye 12 rotating within the patient's eye socket. To make one or more of those determination(s) the control module 18 can employ information defining a predetermined correlation or relationship (which may be generated by the control module 18) between movement of the pupil 32 and a resultant movement of the retina 11, such that by measuring or tracking the pupil's 32 movement and/or position, the control module 18 can determine movement and/or position of an imaging location on the retina 11 based on the pupil movement information.

The control module 18 can also compensate for movements in the patient's eye 12 during image acquisition, in dependence on the determined position or movement of the imaging target, by adjusting an angle of the light beam emitted by the light source 14. For example, the control module 18 can adjust an angle of incidence of the light beam relative to the patient's eye 12 such that, as the patient's eye 12 moves, as tracked by the control module 18 as described above, the light beam tracks the position of the target on the retina 11. The angle of incidence of the light beam that contacts the retina 11 may be adjusted in dependence on the determined movement of the target such that light beam tracks the target. In one example embodiment herein, the angle of incidence of the light beam can be controlled by adjusting the objective optics 15 or by adjusting the angle and position of the light source 14 using a scanning system (not shown) or the like. Adjusting the angle of incidence of the light beam can improve a signal to noise ratio of the resultant target image and furthermore substantially reduce or remove artefacts from generated images caused by movements of the patient's eye 12 during image acquisition.

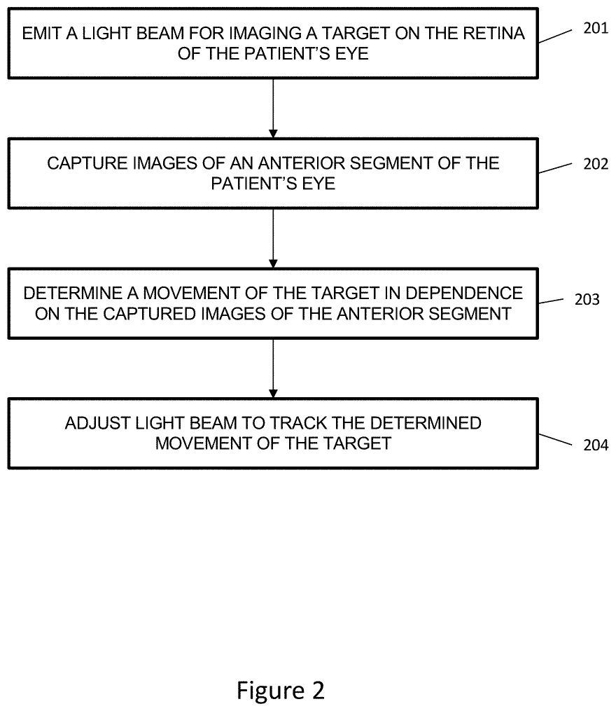

Turning now to FIG. 2 there is shown a flow diagram of a method of compensating for movements of a patient's eye 12 during image acquisition of a target or target location on the posterior segment or retina 11 within a patient's eye 12 according to an example embodiment herein. In Step 201 a light beam for imaging the target is emitted from a light source 14 towards the target on the retina 11. In one example embodiment herein, the light source 14 forms part of an OCT imaging module for acquiring OCT images of a target location in the patient's eye 12 and/or a scanning laser ophthalmoscope, SLO, imaging module for acquiring SLO images of the target location in the patient's eye 12. The light beam may be for imaging a target on the patient's retina 11.

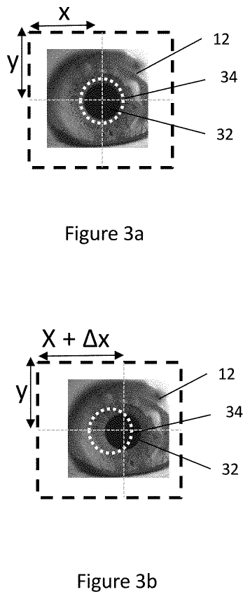

In Step 202 images of the anterior segment 13 of the patient's eye 12 are captured. The images of the anterior segment 13 may be used to track the position and/or movement of the anterior segment 13 of the patient's eye 12. In one example embodiment herein, the position and movement of the patient's pupil 32 (forming at least part of the anterior segment 13) are tracked in Step 202. In more detail, the pupil imager 16 acquires real-time images of the pupil 32 such that movements of the patient's pupil 32 can be monitored and tracked. During image acquisition involuntary eye movements may cause the patient's eye 12 to drift or move relative to a fixation target. Tracking the position of the patient's pupil 32 may comprise detecting a reference pupil position in which the patient is looking directly at the fixation target (see, e.g., FIG. 3a described below). Tracking the movement of the patient's pupil 32 may comprise detecting aberrations or deviations of the pupil's position from the reference pupil position.

In Step 203 a movement of the location of the target is determined in dependence on the captured images of the anterior segment 12 captured in Step 202. For example, a movement of the patient's retina 11 may be determined in dependence on the position and/or movement of the patient's pupil 32 in the captured images. With regards to such movement, as the patient's eye 12 moves within the eye socket the anterior segment 13 of the eye 12 moves to vary the fixation point of the patient which causes a corresponding movement of the retina 11 or posterior segment of the patient's eye 12 as the eye 12 rotates. The movement of the posterior segment of the patient's eye 12 may be a movement of the retina 11 upon which the target is located.

The movement of the target on the patient's retina 11 may be determined (in Step 203) by one or more of: a look-up table comprising retina displacements corresponding to a measured pupil displacement; measuring an axial length between the pupil and the target location on the retina 11 and determining the displacement of the target location in dependence on the measured axial length and tracked pupil movement; and acquiring a series of SLO images of the retina 11 whilst simultaneously tracking the patient's pupil position to create a relationship between tracked pupil position and retina position to calibrate the imaging system 10 prior to commencing an OCT scan, and using that relationship to determine the movement.

In Step 204 the light beam emitted by the light source 14 is adjusted to compensate for the movements of the target as the patient's eye 12 moves. Compensating for movements of the patient's eye 12 may comprise adjusting the angle of incidence of the light beam that focuses on the retina 11 in dependence on the determined movement (from Step 203) of the target such that the light beam tracks or follows the target on the patient's retina 11 in real-time as the patient's eye 12 moves. The angle of the beam emitted from the light source 14 may be controlled by varying the angle and/or position of the light source 14 or by operating a scanning system (not shown) or the objective optics 15 such that the emitted light beam follows the target location within the patient's eye 12.

Turning now to FIGS. 3a and 3b there is shown a series of example images of a patient's eye 12 captured by the pupil imager 16. In FIG. 3a the patient's eye 12 is looking directly at a fixation target within the ophthalmic imaging system 10 such that the patient's pupil 32 is aligned with a pupil reference position 34. When the patient's pupil 32 is located at the pupil reference position 34 the (x, y) coordinates of the pupil 32 are determined by the control module 18 and set as the reference position 34. In one example embodiment herein, the location of the pupil 32 within the captured image is determined by the control module 18 performing predetermined image processing on the images captured by the pupil imager 16 in Step 201.

In FIG. 3b the patient's eye 12 has moved from the pupil reference position 34 to a second position shown in FIG. 3b. As shown in FIG. 3b the patient's pupil 32 is displaced by a displacement Δx along the x-axis and the position of the pupil 32 is unchanged relative to the y-axis. This displacement (movement) is synonymous with the patient moving their gaze to one side of the fixation target. The control module 16 can determine (e.g., in Step 202) the displacement of the patient's pupil 32 by processing the received image data from the pupil imager 16 and determining the location of the patient's pupil 32 in real-time. In cases where the Δx displacement in the pupil image data is a 2D movement the control module 18 assumes that the patient's head is fixed and that a 2D movement of the pupil 32 in the image data is a result of the patient's eye 12 rotating within the eye socket.

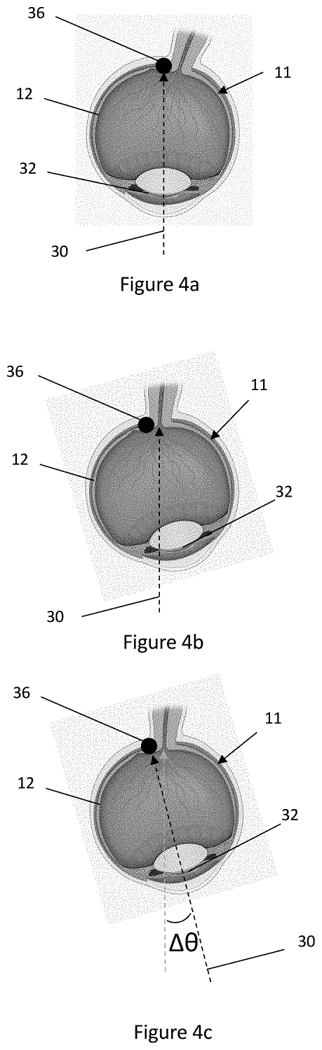

FIG. 4a shows a schematic plan sectional view of the patient's eye 12 in a position corresponding to the reference position 34 shown in FIG. 3a. In FIG. 4a the patient's eye 12 is staring directly at the fixation target (not shown) such that the eye 12 is in the reference position 34. As shown in FIG. 4a when the patient's eye 12 is in the reference position 34, a light beam 30 emitted from the light source 12 passes through the pupil 32 and is incident on the target location 36 on the patient's retina. The target location 36 or location may be a location on the patient's retina 11 to be imaged. For example, the target location 36 may take the form of any one of a point, an area, a region, a line or any other area to be imaged by the imaging system 10.

Turning now to FIG. 4b the patient's eye 12 is shown in the second position corresponding to the position of the pupil 32 in FIG. 3b where the pupil 32 has been displaced from the reference position 34. In FIG. 4b the pupil 32 is displaced by displacement Δx along the x-axis of the 2D image captured by the pupil tracker 16 as a result of the eye 12 rotating within the patient's eye socket. Rotating the eye 12 within the socket causes the pupil 32 to be displaced by displacement Δx with reference to the coordinate system illustrated in FIG. 3b. Furthermore, moving the eye 12 from the pupil reference position 34 to the second position has the effect of moving the target location 36 relative to the light beam 30. As shown in FIG. 4b, the eye 12 has rotated but the light beam 30 has stayed stationary thereby resulting in the light beam 30 no longer being incident on the imaging target 36 on the retina 11.

FIG. 4c represents an example of how the control module 18 compensates (in Step 204) for the movement of the patient's eye 12, by adjusting the angle of incidence of the light beam 30 in real-time such that the light beam 30 tracks the movement of the imaging target 36 within the eye 12. As shown in FIG. 4c the angle of incidence of the light beam 30 has been adjusted by Δθ degrees by the control module 18. The value of Δθ can be determined by the control module 18 in dependence on the tracked position of the anterior segment 13 of the patient's eye 12 (determined in Step 202). The pupil imager 16 captures real-time images of the patient's pupil 32 such that the control module 18 can compensate for movements of the patient's eye 12 by adjusting the angle of incidence of the light beam 30. The control module 18 may use closed loop feedback of the position of the patient's pupil 32 to determine the position of the imaging target and subsequently adjust the light beam such that it is incident on the imaging target 36.

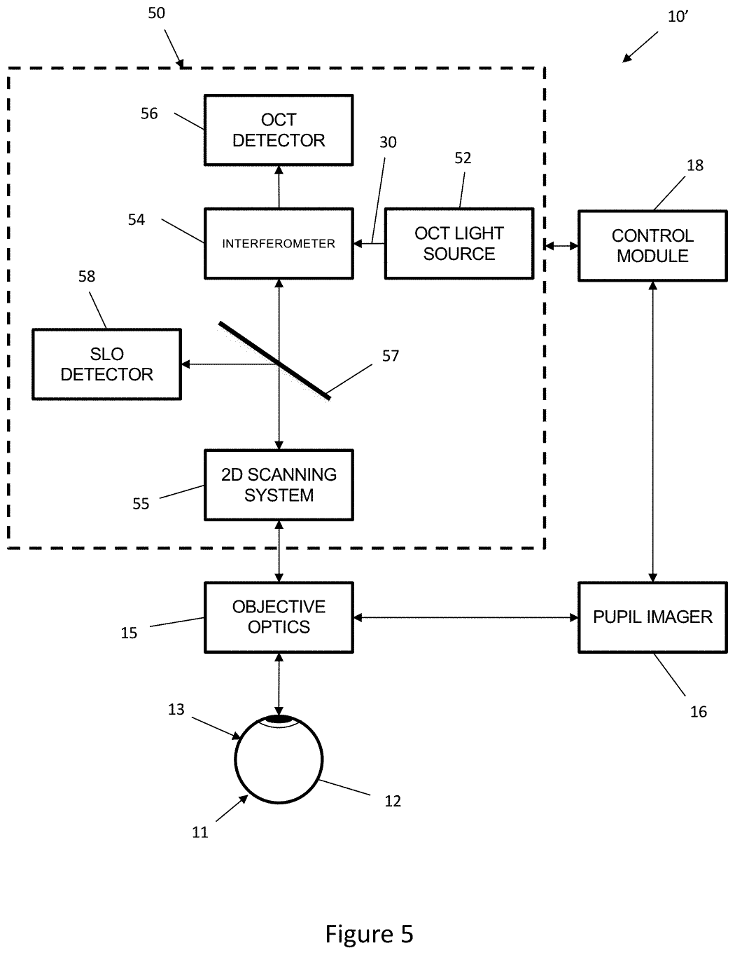

Turning now to FIG. 5 there is shown a further example embodiment of the ophthalmic imaging system 10′. The ophthalmic imaging system 10′ shown in FIG. 5 comprises objective optics 15, a pupil imager 16 and a control module 18, which are like those same corresponding components described above with respect to the ophthalmic imaging system 10 shown in FIG. 1. The ophthalmic imaging system 10′ of FIG. 5 further comprises an optical coherence topography, OCT, imaging module 50 optically coupled to the objective optics 15 and controllable by the control module 18. The OCT imaging module 50 is configured to acquire OCT images of a target location 36 within a posterior segment of the patient's eye 12. For example, the OCT imaging module 50 can acquire OCT images of a target 36 on the patient's retina 11.

As shown in FIG. 5, the OCT imaging module 50 comprises an OCT light source 52 optically coupled to an interferometer 54. The interferometer 54 comprises (or is otherwise coupled to or within) (a) a sample arm (not shown) optically coupled to a 2D scanning system 55 and (b) a reference arm (not shown), as is commonplace in an OCT imaging system. The interferometer 54 is configured to generate an interference signal by combining light beams in the reference arm and the sample arm of the OCT imaging module 50.

The OCT light source 52 is configured to emit a light beam 30 for acquiring OCT images of the target 36 within the patient's eye 12. The OCT light source 52 and interferometer 54 are optically coupled to the 2D scanning system 55, wherein the light source 52 is optically coupled to the 2D scanning system 55 by way of the interferometer 54 and a beam splitter 57. Furthermore, the 2D scanning system 55 is optically coupled to the objective optics 15. The 2D scanning system 55 can be controlled by the control module 18 to adjust the light beam 30 emitted by the light source 52 such that the incident angle of the light beam 30 on the imaging target 36 is adjusted accordingly. For example, and by way of illustration, the 2D scanning system 55 can be operated to scan a patient's retina 11 with the light beam 30 or can be used to adjust the incident angle of the light beam 30 to compensate for detected movements of the patient's eye 12, as described above. The OCT imaging module 50 further comprises an OCT detector 56 for detecting an interference signal from the interferometer 54 resulting from light reflected from the target location within the patient's eye 12 such that OCT images of the patient's eye 12 can be generated by the OCT imaging module 50, for presentation to a user by way of a user interface (not shown).

The OCT imaging module 50 further comprises a scanning laser ophthalmoscopy, SLO, detector 58. A portion of the above-mentioned light reflected from the target 36 on the retina 11 is tapped or split off from the sample arm in the OCT imaging module 50 by the beam splitter 57, and provided to SLO detector 58, and another portion of the reflected light is provided to the interferometer 54 (via beam splitter 57) which operates as described above. The detector 58 generates a SLO image of the patient's retina 11 from received light reflected from the patient's eye 12, wherein the SLO image can be presented to a user by way of a user interface (not shown). In one example embodiment herein, the SLO image of the patient's retina 11 is used as a reference image such that the target location 36 on the patient's retina 11 may be selected based on the SLO reference image of the retina 11. Images and/or other information output by the SLO detector 58 and the OCT detector 56 also can be provided to the control module 18 for processing as described herein.

Furthermore, the SLO image of the retina 11 can be used to determine or calibrate (by, for example and without limitation, control module 18 or another processor) a relationship between a measured pupil movement and a movement of the target 36 as a result of the retina 11 also moving. For example, a series of SLO images can be acquired over a period of time whilst simultaneously tracking the location of the patient's pupil 32 such that the position of the target can be determined for a variety of pupil positions. In one example embodiment herein, the control module 18 determines a relationship between tracked pupil position and target position based on the series of SLO images and pupil positions in the captured images. Also, in one example embodiment herein, the SLO detector 58 is used to acquire SLO images of the retina 11 prior to commencing an OCT scan of the retina 11, although this example is not limiting.

In at least some cases, it might not be possible to simultaneously acquire SLO images and OCT images of the retina 11 using an SLO detector 58 with light tapped off from the main OCT beam owing to the scanning profile required for acquiring an OCT B-scan. Nonetheless, in such cases it is possible to acquire SLO images of the patient's retina 11 prior to commencing an OCT B-scan using light tapped off from the main OCT light beam 30 such that the SLO images can be used to calibrate the system based on a determination of a relationship between the movement of the patient's pupil 32 and a resultant movement of the target location 36 on the retina 11. Furthermore, the SLO image can be used to identify the location of a target for the OCT scan.

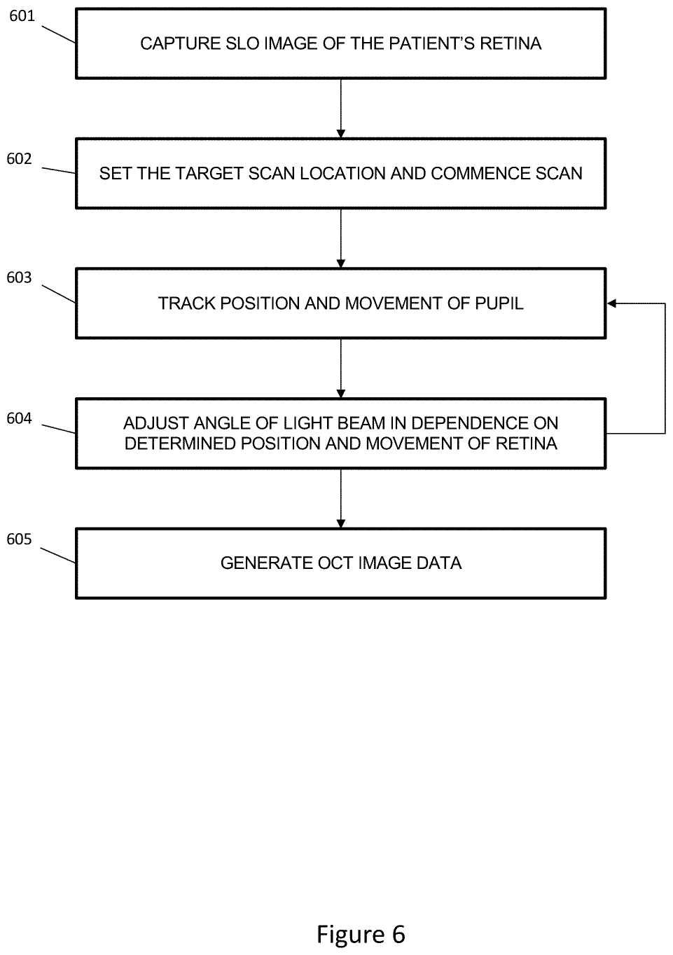

FIG. 6 is a flow diagram of a method according to another example embodiment herein, for imaging a target within a patient's eye 12 using the ophthalmic imaging system 10 comprising the OCT imaging module 50. In Step 601 a scanning laser ophthalmoscopy, SLO, reference image of a posterior segment of the patient's eye 12 or of the retina 11 is captured by the SLO detector 58. In one example embodiment herein, the SLO reference image of the posterior segment (or retina 11) of the patient's eye 12 is an SLO image of the patient's retina 11. Capturing of the SLO image of the posterior segment (or retina 11) of the patient's eye 12 can include and/or be based, at least in part, on splitting of the reflected imaging beam in the sample arm, after which a portion of the split light provided to the detector 58 is detected by the detector 58 to capture/generate the image.

Next, in Step 602 the location of the imaging target 36 on the retina 11 of the patient's eye 12 is determined. In one example embodiment herein, in Step 602 the determining of a scan location for imaging the target 36 on the retina 11 of the patient's eye 12 comprises identifying the target 36 on the SLO reference image and setting the scan location of the light beam 30 in dependence on the identified location. For example, and in more detail, determining the target location 36 on the retina 11 can comprise selecting a target location 36 on the patient's retina 11 from the SLO reference image acquired by the SLO detector 58 in Step 601. The control module 18 can perform image processing on the SLO reference image to identify a target or a clinician may review the acquired SLO reference image and select a target. Once the target scan location has been determined the OCT scan is started and OCT image data of the target location 36 is acquired by the OCT detector 56. The control module 18 can control the 2D scanning system 55 to adjust the light beam from the OCT light source 52 to ensure the light beam 30 is incident on the determined target at the start of the OCT scan when the patient's eye 12 is in the reference location 34.

In Step 603 images of the anterior segment 13 of the patient's eye are captured such that the position and movement of the anterior segment 13 of the patient's eye 12 can be tracked. Tracking the anterior segment 13 of the patient's eye 12 may comprise tracking the position and movement of the patient's pupil 32. Tracking of the anterior segment 13 of the patient's eye 12 may comprise capturing images such as video images of the anterior segment 13 such that the position of the anterior segment 13 can be tracked. An imaging device such as a pupil imager 16 or camera may be used to acquire pupil image data or to capture images of the anterior segment 13 of the patient's eye 12. Tracking the position and movement of the anterior segment 13 of the patient's eye 12 may comprise detecting a pupil reference position 34 in which the patient's pupil 32 is focused on a fixation target. Movements may occur of the patient's pupil 32, such as movements of the pupil 32 from the pupil reference position caused by involuntary movements of the patient's eye 12. The involuntary movements of the patient's eye 12 may be a result of the eye 12 moving within the patient's eye socket or orbit. Images of the eye 12 are captured such that movements and positions of the pupil 32 (anterior segment 13) can be determined and tracked.

In Step 604 the angle of the light beam 30 emitted from the OCT light source 52 is adjusted in dependence on the tracked position and/or movement(s) of the anterior segment 13 of the patient's eye 12, determined in Step 603 from the captured images. Adjusting the angle of the OCT light beam in dependence on the tracked position and/or movement of the patient's pupil 32 beneficially compensates for involuntary movements of the patient's eye 12 by following the imaging target 36 on the retina 11 of the patient's eye 12 with the light beam 30. Step 604 further comprises determining a position and movement of the target and/or the retina 11 in dependence on the tracked position and movement of the pupil 32. In an example embodiment herein, tracking the position and movement(s) of the pupil 32 can allow the position and movement of the target and/or retina 11 to be determined.

Adjusting the angle of the light beam 30 to compensate for movements of the patient's eye 12 forms part of a closed-loop feedback system. The position and movement of the patient's pupil 32 is tracked in real-time in Step 603 and the angle of incidence of the OCT light beam is adjusted in Step 604 in dependence on the tracked pupil position and/or movement such that the light beam follows the imaging target location 36, according to an example embodiment herein. In Step 605 OCT image data of the imaging target 36 is generated based on a detection made by the OCT detector 56 of light reflected from the target, after the adjusting of the light beam 30 was made in Step 604.

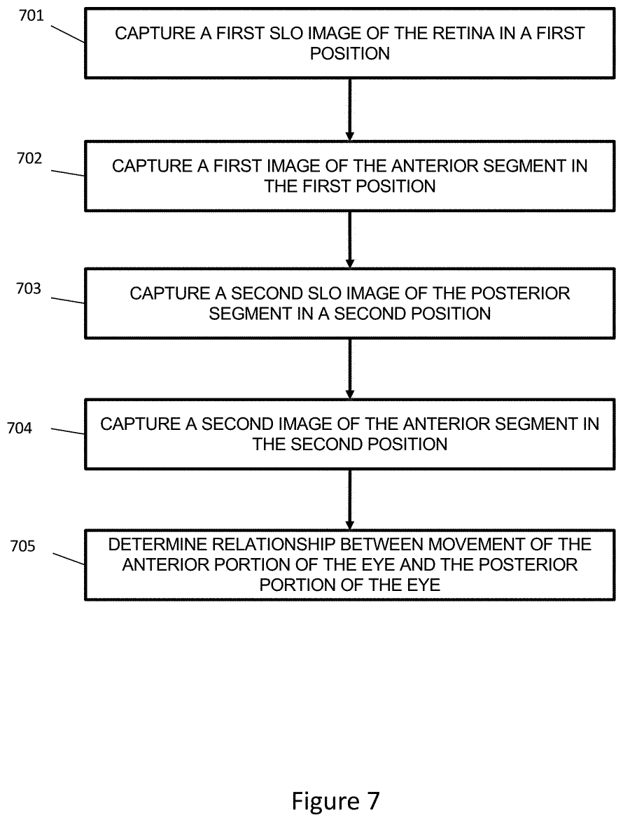

Turning now to FIG. 7 there is shown a flow diagram of a method of determining a relationship between movement of the anterior segment 13 of the patient's eye 12 and a resultant movement of the posterior segment 11 of the patient's eye 12 according to another example embodiment herein. In Step 701 a first SLO image of the retina 11 is captured by the SLO detector 58 when the patient's eye 12, and thus retina 11, is in a first position. The first position may be a reference position 34, for example, a position in which the patient's eye 12 is staring (focusing) directly at a fixation target (see, e.g., FIG. 3a).

In Step 702 a first image of the anterior segment 13 of the patient's eye 12 in the first position is captured using, for example, the pupil imaged 16. The first image of the anterior segment 13 can be captured simultaneously with the first SLO image of the retina 11. The first image of the anterior segment 13 of the patient's eye 12 is used to detect the position of the patient's pupil 32 when the eye 12 is in the first position.

In Step 703 a second SLO image of the retina 11 is captured with the eye 12, and thus retina 11, in a second position. The second position is a position in which the patient has moved their eye 12 through either an involuntary eye movement or by following a fixation target such that the eye 12 is articulated whilst the patient's head is in a fixed position. In Step 704 a second pupil image of the anterior segment 13 is acquired while the patient's eye 12 in the second position.

In Step 705 a relationship between the tracked movement of the anterior segment 13 of the patient's eye 12 and a resultant movement of the target and/or retina 11 is determined. For example, in Step 705 the position of the anterior segment 13, and in particular the position of the pupil 32, is determined in the first and second pupil images acquired by the pupil imager 16. Also, movement of the pupil 32 from the first position to the second position may be determined from the first and second images captured by the pupil imager 16.

Also in Step 705, the movement of the retina 11, and thus target location 36, is determined from the first and second SLO images. Determining the movement of the retina 11 from the first position to the second position comprises, in one example embodiment herein, tracking the movement of an anatomical feature or landmark on the patient's retina 11 shown on the first and second SLO image.

Then, as part of Step 705 the above-mentioned relationship between tracked movement of the patient's pupil 32 or anterior segment 13 of the eye 12 and the determined movement of the retina 11 is determined by comparing the detected movement of the retina 11 with the detected movement of the anterior segment 13. That relationship can be used to determine a movement of the target location 36 on the retina 11 based on tracked movement of the patient's pupil 32, in one or more of the method(s) described above.

In one example embodiment herein, the method can be repeated with further SLO images and images of the anterior segment 11 of the patient's eye 12 in varying positions to improve the accuracy of the determined relationship between the tracked position of the patient's pupil 32 and the resultant location of the imaging target 36 on the patient's retina 11. For example, a series of SLO images and pupil images can be captured when the patient is looking directly at a fixation target and involuntary eye movements can be tracked. Furthermore, eye steering can be performed to steer the patient's eye 12 through various positions in which SLO images and pupil images are captured to determine the relationship between pupil movement and movement of the imaging target on the patient's retina 11.

In another example embodiment herein, an axial length of the patient's eye 12 can be measured from the patient's pupil 32 to the retina 11, to enable movement(s) of the target 36 on the patient's retina 11 to be determined in dependence on the tracked position of the patient's pupil 32. For example, if a displacement of the patient's pupil 32 due to the eye 12 rotating within the eye socket is known the resultant movement of the target location 36 on the patient's retina 11 can also be determined. This in turn allows the imaging beam to be adjusted such that the incident angle of the imaging beam may be adjusted in real time to track the imaging target on the patient's retina 11. The axial length of the patient's eye 12 may be measured using the OCT imaging module 50 to perform biometry measurements on the patient's eye 12. The biometry measurements may include measuring the axial length of the patient's eye 12.

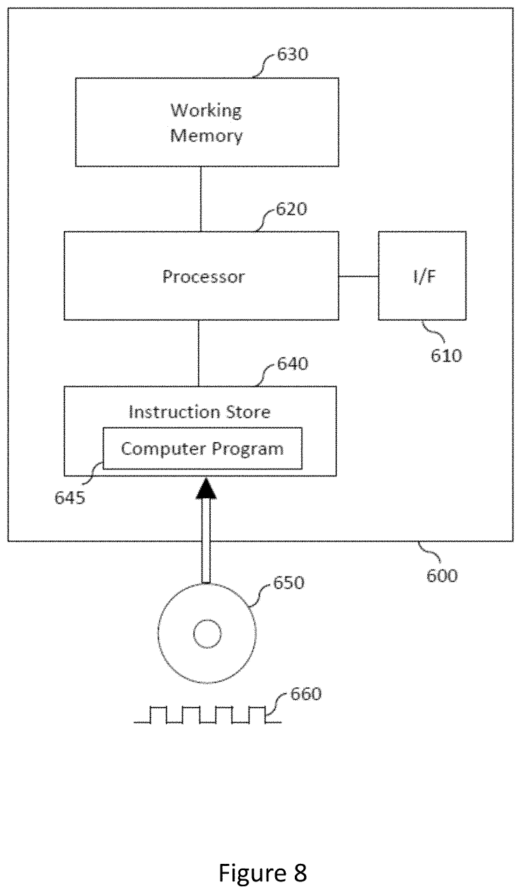

FIG. 8 is a schematic illustration of a programmable signal processing hardware 600, configured to image a target or a location on a patient's retina and/or to compensate for movements in a patient's eye during retinal imaging. The programmable signal processing hardware 600 can perform the functionalities of the control module 18, and, in one example embodiment herein, at least part of the hardware 600 is included in the control module 18. The programmable signal processing apparatus 600 comprises a communication interface (I/F) 610, for receiving pupil image data from the pupil imager 16, SLO image data from the SLO detector 58, OCT image data from the OCT detector 56, and scanning location data from the 2D scanning system 55, and for outputting determined position(s) and/or movement(s) of an imaging target and of applicable parts of the eye, and/or for outputting light beam and/or light source positional and/or angular information. In one example embodiment herein, the communication interface (I/F) 610 can input/output any information obtained as part of the methods described herein.

The signal processing apparatus 600 further comprises a processor (e.g. a Central Processing Unit, CPU, and/or a Graphics Processing Unit, GPU) 620, a working memory 630 (e.g. a random access memory) and an instruction store 640 storing a computer program 645 comprising computer-readable instructions which, when executed by the processor 620, cause the processor 620 to perform various functions including those of the control module 18 and/or the functions of the methods described herein. In one example embodiment herein, only the processor 620 is included in the control module 18, although in other examples one or more additional components of the hardware 600 also are included in the control module 18 as well.

The working memory 630 stores information used by the processor 620 during execution of the computer program 645. The instruction store 640 comprises, for example, a ROM (e.g. in the form of an electrically erasable programmable read-only memory (EEPROM) or flash memory) which is pre-loaded with the computer-readable instructions. Alternatively, the instruction store 640 comprises a RAM or similar type of memory, and the computer-readable instructions of the computer program 645 can be input thereto from a computer program product, such as a non-transitory, computer-readable storage medium 650 in the form of a CD-ROM, DVDROM, etc. or a computer-readable signal 660 carrying the computer-readable instructions. In any case, the computer program 645, when executed by the processor 620, causes the processor 620 to perform the methods described herein, including by example and without limitation, a method of imaging a patient's retina and/or a method of compensating for movements of a patient's eye during retinal imaging as described hereinabove. In one example embodiment herein, the control module 18 of the example embodiments described above comprises the computer processor 620 and memory 640 storing the computer-readable instructions which, when executed by the computer processor 620, cause the computer processor 620 to perform the methods described herein, including by example and without limitation, a method of imaging a patient's retina and/or a method of compensating for movements of a patient's eye during retinal imaging as described herein.

In the foregoing description, example aspects are described with reference to several example embodiments. Accordingly, the specification should be regarded as illustrative, rather than restrictive. Similarly, the figures illustrated in the drawings, which highlight the functionality and advantages of the example embodiments, are presented for example purposes only. The architecture of the example embodiments is sufficiently flexible and configurable, such that it may be utilized in ways other than those shown in the accompanying figures.

Software embodiments of the examples presented herein may be provided as, a computer program, or software, such as one or more programs having instructions or sequences of instructions, included or stored in an article of manufacture such as a machine-accessible or machine-readable medium, an instruction store, or computer-readable storage device, each of which can be non-transitory, in one example embodiment. The program or instructions on the non-transitory machine-accessible medium, machine-readable medium, instruction store, or computer-readable storage device, may be used to program a computer system or other electronic device. The machine- or computer-readable medium, instruction store, and storage device may include, but are not limited to, floppy diskettes, optical disks, and magneto-optical disks or other types of media/machine-readable medium/instruction store/storage device suitable for storing or transmitting electronic instructions. The techniques described herein are not limited to any particular software configuration. They may find applicability in any computing or processing environment. The terms “computer-readable”, “machine-accessible medium”, “machine-readable medium”, “instruction store”, and “computer-readable storage device” used herein shall include any medium that is capable of storing, encoding, or transmitting instructions or a sequence of instructions for execution by the machine, computer, or computer processor and that causes the machine/computer/computer processor to perform any one of the methods described herein. Furthermore, it is common in the art to speak of software, in one form or another (e.g., program, procedure, process, application, module, unit, logic, and so on), as taking an action or causing a result. Such expressions are merely a shorthand way of stating that the execution of the software by a processing system causes the processor to perform an action to produce a result.

Some embodiments may also be implemented by the preparation of application-specific integrated circuits, field-programmable gate arrays, or by interconnecting an appropriate network of conventional component circuits.

Some embodiments include a computer program product. The computer program product may be a storage medium or media, instruction store(s), or storage device(s), having instructions stored thereon or therein which can be used to control, or cause, a computer or computer processor to perform any of the procedures of the example embodiments described herein. The storage medium/instruction store/storage device may include, by example and without limitation, an optical disc, a ROM, a RAM, an EPROM, an EEPROM, a DRAM, a VRAM, a flash memory, a flash card, a magnetic card, an optical card, nanosystems, a molecular memory integrated circuit, a RAID, remote data storage/archive/warehousing, and/or any other type of device suitable for storing instructions and/or data.

Stored on any one of the computer-readable medium or media, instruction store(s), or storage device(s), some implementations include software for controlling both the hardware of the system and for enabling the system or microprocessor to interact with a human user or other mechanism utilizing the results of the example embodiments described herein. Such software may include without limitation device drivers, operating systems, and user applications. Ultimately, such computer-readable media or storage device(s) further include software for performing example aspects of the invention, as described above.

Included in the programming and/or software of the system are software modules for implementing the procedures described herein. In some example embodiments herein, a module includes software, although in other example embodiments herein, a module includes hardware, or a combination of hardware and software.

While various example embodiments of the present invention have been described above, it should be understood that they have been presented by way of example, and not limitation. It will be apparent to persons skilled in the relevant art(s) that various changes in form and detail can be made therein. Thus, the present invention should not be limited by any of the above described example embodiments, but should be defined only in accordance with the following claims and their equivalents.

It is also to be understood that any procedures recited in the claims need not be performed in the order presented.

While this specification contains many specific embodiment details, these should not be construed as limitations on the scope of any inventions or of what may be claimed, but rather as descriptions of features specific to particular embodiments described herein. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

In certain circumstances, multitasking and parallel processing may be advantageous. Moreover, the separation of various components in the embodiments described above should not be understood as requiring such separation in all embodiments, and it should be understood that the described program components and systems can generally be integrated together in a single software product or packaged into multiple software products.

Having now described some illustrative embodiments and embodiments, it is apparent that the foregoing is illustrative and not limiting, having been presented by way of example. In particular, although many of the examples presented herein involve specific combinations of apparatus or software elements, those elements may be combined in other ways to accomplish the same objectives. Acts, elements and features discussed only in connection with one embodiment are not intended to be excluded from a similar role in other embodiments or embodiments.

Some of the embodiments described above are summarised in the following examples E1 to E24:
E1. An ophthalmic imaging system for imaging a location on a retina of a patient's eye, the ophthalmic imaging system comprising:
   a light source arranged to emit a light beam towards the location on the retina;
   an imaging device arranged to capture images of an anterior segment of the patient's eye; and
   a controller arranged to determine a movement of the location based on the captured images of the anterior segment of the patient's eye;
   wherein the controller is further arranged to adjust an angle of incidence of the light beam directed towards the retina based on the determined movement of the location.
E2. An ophthalmic imaging system according to E1, further comprising an optical coherence tomography, OCT, imaging module comprising:
   an interferometer arranged to generate an interference signal from a reflected light beam received from the location on the retina via a sample arm of the OCT imaging module; and
   an OCT detector for generating OCT image data indicative of the location based on the generated interference signal.
E3. An ophthalmic imaging system according to E2, further comprising a scanning laser ophthalmoscopy, SLO, detector arranged to receive a portion of the reflected light beam from the sample arm to generate SLO image data of the retina.
E4. An ophthalmic imaging system according to E3, comprising a beam splitter located in the sample arm arranged to split the portion of the reflected light beam from the reflected light beam in the sample arm before the reflected light beam enters the interferometer.
E5. An ophthalmic imaging system according to E3 or E4, wherein the controller is arranged to set a scan location for the light beam to image the target in dependence on the generated SLO image data.
E6. An ophthalmic imaging system according to any one of E3 to E5, wherein the controller is arranged to acquire a first SLO image of the retina in a first position and a second SLO image of the retina in a second position from the SLO detector; and
   wherein the controller is further configured to track a position of the anterior segment of the eye; and
   determine a relationship between the tracked position of the anterior segment of the eye and a position of the retina by comparing the first and second SLO images with the tracked position of the anterior segment of the eye.
E7. An ophthalmic imaging system according to E6, wherein the controller is arranged to compare a movement of an anatomical feature from a first position in the first SLO image to a second position in the second SLO image, with the tracked position of the anterior segment of the eye to determine the relationship between the position of the anterior segment of the eye and the retina.
E8. An ophthalmic imaging system according to any one of E1 to E7, wherein the controller is arranged to track movements of the anterior segment of the patient's eye in dependence on the captured images.
E9. An ophthalmic imaging system according to any one of E1 to E8, wherein the imaging device is a camera.
E10. An ophthalmic imaging system according to any one of E1 to E9, wherein the anterior segment of the patient's eye is the patient's pupil.
E11. A method of compensating for movements of a patient's eye during imaging of a location on a retina of the patient's eye, the method comprising:
   capturing images of an anterior segment of the patient's eye;
   determining a movement of the location based on the captured images of the anterior segment; and
   adjusting an angle of incidence of a light beam directed to the retina for imaging the location on the retina based on the determined movement of the location to compensate for movements of the patient's eye during imaging.
E12. A method according to E11, further comprising:
   acquiring a first scanning laser ophthalmoscopy, SLO, image of the retina in a first position;
   acquiring a second SLO image of the retina in a second position;
   tracking a position of the anterior segment of the eye; and
   determining a relationship between the tracked position of the anterior segment of the eye and a position of the retina by comparing the first and second SLO images with the tracked position of the anterior segment of the eye.
E13. A method according to E12, wherein comparing the first and second SLO images with the tracked position of the anterior segment comprises comparing a movement of an anatomical feature from a first position in the first SLO image to
   a second position in the second SLO image with the tracked position of the anterior segment of the eye to determine the relationship between the tracked position of the anterior segment of the eye and the retina.
E14. A method according to E12 or E13, comprising moving a fixation target from a first fixation position to a second fixation position to steer the patient's eye to move the retina from the first position to the second position.
E15. A method according to any one of E11 to E14, comprising acquiring a reference SLO image of the retina of the patient's eye and selecting a scan position for imaging the location on the retina based on the acquired reference SLO image.
E16. A method according to E15, wherein acquiring the reference SLO image comprises splitting a reflected light beam received from the location on the retina.
E17. A method according to E16, wherein splitting the reflected light beam comprises splitting the reflected light beam in a sample arm of an optical coherence topography imaging module.
E18. A method according to E17, wherein splitting the reflected light beam comprises splitting the reflected light beam prior to the reflected light beam entering an interferometer.
E19. A method according to any one of E11 to E18, wherein capturing images of the anterior segment comprises detecting a reference position of the patient's eye and detecting a movement of the anterior segment from the reference position.
E20. A method according to any one of E11 to E19, wherein determining a movement of the location comprises tracking the position of the anterior segment and comparing the tracked position of the anterior segment with a look-up table to determine the movement of the location.
E21. A method according to any one of E11 to E20, comprising measuring an axial length between the anterior segment and the retina of the patient's eye and determining a relationship between the captured images of the patient's eye and the movement of the location in dependence on the measured axial length.
E22. A method according to any one of E11 to E21, comprising performing closed-loop feedback to adjust the angle of incidence of the light beam based on the determined movement of the location.
E23. A method according to any one of E11 to E22, wherein determining the movement of the location comprises processing the captured images of the anterior segment of the eye to track a position of the patient's pupil.
E24. A method according to E23, wherein processing the captured images to track the position of the patient's pupil is performed in real-time.

1. An ophthalmic imaging system for imaging a location on a retina of a patient's eye, the ophthalmic imaging system comprising:
a light source arranged to emit a light beam towards the location on the retina;
an imaging device arranged to capture images of an anterior segment of the patient's eye; and
a controller arranged to determine a movement of the location based on the captured images of the anterior segment of the patient's eye;
wherein the controller is further arranged to adjust an angle of incidence of the light beam directed towards the retina based on the determined movement of the location.

2. An ophthalmic imaging system as claimed in claim 1, further comprising an optical coherence tomography, OCT, imaging module comprising:
an interferometer arranged to generate an interference signal from a reflected light beam received from the location on the retina via a sample arm of the OCT imaging module; and
an OCT detector for generating OCT image data indicative of the location based on the generated interference signal.

3. An ophthalmic imaging system as claimed in claim 2, further comprising a scanning laser ophthalmoscopy, SLO, detector arranged to receive a portion of the reflected light beam from the sample arm to generate SLO image data of the retina.

4. An ophthalmic imaging system as claimed in claim 3, comprising a beam splitter located in the sample arm arranged to split the portion of the reflected light beam from the reflected light beam in the sample arm before the reflected light beam enters the interferometer.

5. An ophthalmic imaging system as claimed in claim 3, wherein the controller is arranged to set a scan location for the light beam to image the target in dependence on the generated SLO image data.

6. An ophthalmic imaging system as claimed in claim 3, wherein the controller is arranged to acquire a first SLO image of the retina in a first position and a second SLO image of the retina in a second position from the SLO detector; and
wherein the controller is further configured to track a position of the anterior segment of the eye; and
determine a relationship between the tracked position of the anterior segment of the eye and a position of the retina by comparing the first and second SLO images with the tracked position of the anterior segment of the eye.

7. An ophthalmic imaging system as claimed in claim 6, wherein the controller is arranged to compare a movement of an anatomical feature from a first position in the first SLO image to a second position in the second SLO image, with the tracked position of the anterior segment of the eye to determine the relationship between the position of the anterior segment of the eye and the retina.

8. An ophthalmic imaging system as claimed in claim 1, wherein the controller is arranged to track movements of the anterior segment of the patient's eye in dependence on the captured images.

9. An ophthalmic imaging system for imaging a location on a retina of a patient's eye, the ophthalmic imaging system comprising:
an ophthalmic coherence tomography, OCT, imaging module arranged to acquire OCT images of the location by emitting a light beam towards the location on the retina;
an imaging device arranged to capture images of the patient's pupil;
a scanning laser ophthalmoscope, SLO, imaging detector arranged to acquire a SLO image of the patient's retina; and
a controller arranged to:
direct the light beam towards the location based on the generated SLO image such that the light beam is incident on the location on the retina;
determine a movement of the location based on the captured images of the patient's pupil; and
adjust an angle of incidence of the light beam directed towards the retina based on the determined movement of the location.

10. A method of compensating for movements of a patient's eye during imaging of a location on a retina of the patient's eye, the method comprising:
capturing images of an anterior segment of the patient's eye;
determining a movement of the location based on the captured images of the anterior segment; and
adjusting an angle of incidence of a light beam directed to the retina for imaging the location on the retina based on the determined movement of the location to compensate for movements of the patient's eye during imaging.

11. A method as claimed in claim 10, further comprising:
acquiring a first scanning laser ophthalmoscopy, SLO, image of the retina in a first position;
acquiring a second SLO image of the retina in a second position;
tracking a position of the anterior segment of the eye; and
determining a relationship between the tracked position of the anterior segment of the eye and a position of the retina by comparing the first and second SLO images with the tracked position of the anterior segment of the eye.

12. A method as claimed in claim 11, wherein comparing the first and second SLO images with the tracked position of the anterior segment comprises comparing a movement of an anatomical feature from a first position in the first SLO image to a second position in the second SLO image with the tracked position of the anterior segment of the eye to determine the relationship between the tracked position of the anterior segment of the eye and the retina.

13. A method as claimed in claim 12, comprising moving a fixation target from a first fixation position to a second fixation position to steer the patient's eye to move the retina from the first position to the second position.

14. A method as claimed in claim 10, comprising acquiring a reference SLO image of the retina of the patient's eye and selecting a scan position for imaging the location on the retina based on the acquired reference SLO image.

15. A method as claimed in claim 14, wherein acquiring the reference SLO image comprises splitting a reflected light beam received from the location on the retina.

16. A method as claimed in claim 15, wherein splitting the reflected light beam comprises splitting the reflected light beam in a sample arm of an optical coherence topography imaging module.

17. A method as claimed in claim 16, wherein splitting the reflected light beam comprises splitting the reflected light beam prior to the reflected light beam entering an interferometer.

18. A method as claimed in claim 10, wherein capturing images of the anterior segment comprises detecting a reference position of the patient's eye and detecting a movement of the anterior segment from the reference position.

19. A method as claimed in claim 10, wherein determining a movement of the location comprises tracking the position of the anterior segment and comparing the tracked position of the anterior segment with a look-up table to determine the movement of the location.

20. A method as claimed in claim 10, comprising measuring an axial length between the anterior segment and the retina of the patient's eye and determining a relationship between the captured images of the patient's eye and the movement of the location in dependence on the measured axial length.
